(19) United States
(12) Patent Application Publication
(10) Pub. No.: US 2026/0123886 A1
(43) Pub. Date: May 7, 2026

(54) CONFIGURABLE IMPLANTABLE MEDICAL DEVICES

(71) Applicant: Cardiac Pacemakers, Inc., St Paul, MN (US)

(72) Inventors: Jonathan H. Kelly, Woodbury, MN (US); Deepa Mahajan, North Oaks, MN (US); Dan Constantin Luca, Plymouth, MN (US); Scott R. Vanderlinde, Plymouth, MN (US)

(21) Appl. No.: 19/379,734

(22) Filed: Nov. 4, 2025

Related U.S. Application Data
(60) Provisional application No. 63/716,889, filed on Nov. 6, 2024.

(51) Int. Cl. A61B 5/00; A61B 5/29

(52) U.S. Cl. CPC A61B 5/686; A61B 5/29; A61B 5/7282; A61B 2560/0468

(57) ABSTRACT

Systems, methods, and devices include approaches for receiving physiological data associated with detected cardiac events from a medical device being operated in a default mode, receiving a command to reprogram the medical device to operate in a non-default mode, and programming the implantable medical device, using a remote device, to operate in the non-default mode. The programming increases an amount of physiological data or number of cardiac events wirelessly transmitted by the medical device compared to the default mode.

### CROSS REFERENCE TO RELATED APPLICATION

This application claims priority to Provisional Application No. 63/716,889, filed Nov. 6, 2024, which is herein incorporated by reference in its entirety.

### TECHNICAL FIELD

The present disclosure generally relates to approaches for remote programming of medical devices used for sensing and monitoring cardiac activity.

### BACKGROUND

Medical devices that allow physicians to monitor cardiac activity are becoming increasingly common in diagnosing and treating medical conditions in patients. Cardiac monitoring can be used, for example, to identify abnormal cardiac rhythms, so that critical alerts can be provided to patients, physicians, or other care providers and so that patients can be treated as needed.

### SUMMARY

In Example 1, a method includes receiving physiological data associated with detected cardiac events from a medical device (e.g., an implantable medical device) being operated in a default mode, receiving a command to reprogram the medical device to operate in a non-default mode, and programming the implantable medical device, using a remote device, to operate in the non-default mode. The programming increases an amount of physiological data wirelessly transmitted by the medical device compared to the default mode.

In Example 2, the method of Example 1, wherein the programming increases a length of physiological data stored for each detected cardiac event.

In Example 3, the method of Examples 1 or 2, wherein the programming increases a frequency at which the physiological data is wirelessly transmitted.

In Example 4, the method of any of Examples 1-3, wherein the physiological data is deleted from memory after being wirelessly transmitted by the implantable medical device.

In Example 5, the method of any of Examples 1-4, wherein the programming adjusts prioritization of storage and deletion of the physiological data.

In Example 6, the method of any of Examples 1-5, wherein the programming increases a maximum number of cardiac events stored to memory.

In Example 7, the method of any of Examples 1-6, wherein the physiological data is wirelessly transmitted to the remote device.

In Example 8, the method of any of Examples 1-7, wherein the programming changes a trigger for initiating transmission of the physiological data.

In Example 9, the method of any of Examples 1-8, wherein the programming changes an amount of time stored to memory before an onset of a cardiac event and an end of the cardiac event.

In Example 10, the method of any of Examples 1-9, wherein the programming increases diagnostic data associated with each cardiac event.

In Example 11, the method of any of Examples 1-10, further comprising:
programming the implantable medical device to revert back to the default operating mode after a predetermined period of time.

In Example 12, the method of any of Examples 1-11, wherein the programming comprises wirelessly transmitting an instruction to the implantable medical device, wherein the instruction causes the implantable medical device to operate on the non-default mode.

In Example 13, a computer program product comprising instructions to cause one or more processors to carry out the steps of the method of Examples 1-12.

In Example 14, a computer-readable medium having stored thereon the computer program product of Example 13.

In Example 15, a mobile computing device comprising the computer-readable medium of Example 14 and comprising a user interface.

In Example 16, a system includes a programmer with a user interface, memory, and one or more processors. The memory stores instructions that, when executed by the one or more processors, cause the programmer to: (1) receive a command to reprogram an implantable medical device to change operating from a default mode to a non-default mode and (2) cause the implantable medical device to be programmed to operate in the non-default mode in response to the command. The non-default mode increases an amount of physiological data wirelessly transmitted by the implantable medical device compared to the default mode.

In Example 17, the system of Example 16, wherein, in the non-default mode, a length of physiological data stored for each detected cardiac event is increased.

In Example 18, the system of Example 16, wherein, in the non-default mode, a frequency at which the physiological data is wirelessly transmitted is increased.

In Example 19, the system of Example 18, wherein the frequency is greater than twice per 24-hour period.

In Example 20, the system of Example 16, wherein the non-default mode changes an amount of time stored to memory before an onset of a cardiac event and an end of the cardiac event.

In Example 21, the system of Example 20, wherein the amount of time is 45 seconds to 2 minutes.

In Example 22, the system of Example 16, further including the implantable medical device, which includes electrodes.

In Example 23, the system of Example 16, further including a remote computing system. The command is initiated at the remote computing system.

In Example 24, in any of Examples 1-23, wherein, in the default mode, the medical device is programmed to transfer physiological data only once or twice per day.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of a system that includes a medical device, in accordance with certain instances of the present disclosure.

FIGS. 2 and 3 show different views of a medical device, in accordance with certain instances of the present disclosure.

FIG. 4 shows a block diagram depicting an illustrative method, in accordance with certain instances of the disclosure.

FIG. 5 is a block diagram depicting an illustrative computing device, in accordance with instances of the disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Medical devices can be equipped with one or more sensing components (e.g., sensors, electrodes) and programmed to sense physiological data such as electrocardiogram (ECG) data. To collect physiological data, one or more medical devices (e.g., implantable cardiac monitors/recorders, external cardiac /nitors/ recorders) can be implanted in or coupled to the patient such that the medical devices can sense the physiological data.

Although such medical devices may attempt to continuously sense physiological data such as ECG data, to save power and memory capacity the medical devices may not ultimately record and then transmit all the sensed physiological data outside of the medical devices. Instead, the sensed physiological data may only be recorded and then transmitted in certain situations. For example, the medical devices may be programmed to process and analyze the sensed physiological data to determine the occurrence of certain cardiac events such as tachycardia events, bradycardia events, pause events, atrial fibrillation events, episodes containing premature ventricular contractions (PVCs), etc. If it is determined that an event has occurred, certain sensed physiological data can be recorded to longer-term memory of the medical device and then later transmitted to another device separate or remote from the medical device. However, recording and transmitting physiological data every time an event occurs can be an inefficient use of the medical device's power (e.g., battery capacity). Further, the medical device may have a limited overall memory capacity due to power and space restrictions.

As such, in a default operating mode, a medical device may utilize various power-saving and memory-saving approaches. One approach is to limit how often physiological data is transmitted from the medical device to an external device. For example, to save power, physiological data may only be transferred once per day. Another approach is to identify the highest priority events and only transmit physiological data associated with the highest priority events. Another approach is to limit how much data (e.g., physiological data, metadata) is saved to memory for each detected event. In some instances, all of these approaches may be used when the medical device is using a default operating mode.

However, these approaches can limit the fidelity of the assessment of the patient and can delay the assessment—and can delay the ultimate prescription and application of therapy to try to address the underlying cause of the detected events. These can be issues, in particular, for patients that have recently changed a drug regimen, have recently been involved in a procedure such as an ablation procedure, or recently received a stent, among other scenarios.

Certain instances of the present disclosure are accordingly directed to approaches for programming or reconfiguring medical devices.

### Cardiac Event Evaluation System

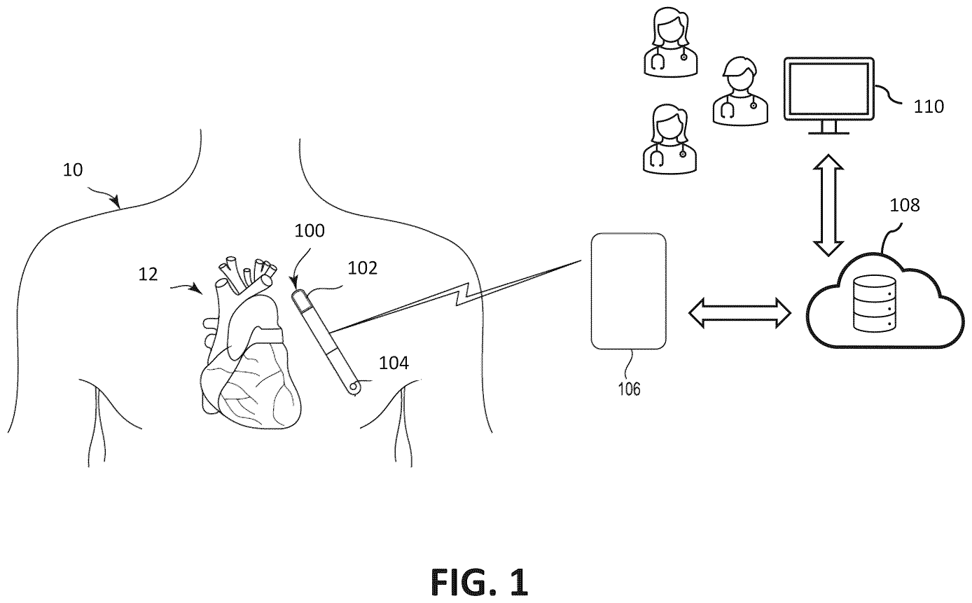

FIG. 1 is a schematic illustration of a medical device 100 within a cardiac event evaluation system. The medical device 100 can be implanted subcutaneously within an implantation location or pocket in the chest or abdomen of the patient 10 and may be configured to sense physiological signals associated with the patient's heart 12. The medical device 100 may be an implantable cardiac monitor (e.g., an implantable diagnostic monitor, an implantable loop recorder) configured and programmed to record physiological parameters such as, for example, one or more cardiac activation signals, heart sounds, blood pressure measurements, oxygen saturations. Although the medical device 100 of FIG. 1 is shown as an implantable cardiac monitor, the approaches described herein can be used in connection with other types of implantable medical devices such as a pulse generator (e.g., pacemaker, defibrillator). Further, although the medical device 100 of FIG. 1 is shown as a medical device that is implanted into the patient 10, the approaches described herein can be used in connection with an external medical device such as a monitoring device that is coupled to a patient's skin or a mobile device that includes one or more sensors for detecting physiological data and/or that can process detected physiological data.

In the example of FIG. 1, the medical device 100 includes electrodes 102 and 104, which sense physiological signals (e.g., electrical cardiac activation signals) of the heart 12. The sensed physiological signals can be used to generate electrocardiogram (ECG) data such as ECG waveforms, which represent sensed cardiac electric activity over time. ECG waveforms can represent measured voltage amplitudes over time and, therefore, may be considered to be time-series cardiac data.

As described in more detail below, certain physiological data can be communicated (e.g., communicated wirelessly using antennas) to a different component within the system. For example, physiological data can be communicated from the medical device 100 to a receiver 106, a computing system 108, and/or a remote computing device 110. The receiver 106 can be a device that is capable of programming, controlling, monitoring, and/or otherwise communicating with the medical device 100. The receiver 106 can help facilitate communication from the medical device 100 to and from another device or system such as the computing system 108 (e.g., laptop computer, desktop computer, server). The receiver 106 and/or the computing system 108 can be communicatively coupled to another computing system 110 with a display on which users (e.g., patients, physicians, technicians) can view data sensed and recorded by the medical device 100. In certain instances, the receiver 106 is a mobile computing device such as a programmer, smartphone, tablet computer, laptop computer, etc. The receiver 106 can operate an application (e.g., a software application used on a smartphone, tablet, and the like) that enables the receiver 106 to communicate with the medical device 100, the computing system 108, and/or the remote computing device 110. In certain instances, the application can enable the receiver 106 to reprogram the medical device 100, as described further herein.

The medical device 100 can be programmed to initially store sensed physiological data such as ECG data to temporary memory such as a buffer or cache memory. But temporary memory can have limited storage capacity. To deal with the limited storage capacity, the medical device 100 can be programmed to cause physiological data to be repeatedly deleted as the amount of stored ECG data reaches the maximum storage capacity of the temporary memory. More specifically, the temporary memory can be operated as a first-in-first-out (FIFO) buffer or circular buffer that is operated to repeatedly delete the oldest data in favor of storing the newest data. However, under certain conditions, physiological data can be transferred and recorded to longer-term memory. Some or all of the physiological data recorded to longer-term memory can then be periodically transmitted to another device (e.g., the receiver 106, the computing system 108, and/or the remote computing device 110). And once the physiological data is transmitted to another device, that physiological data can be deleted from the longer-term memory so that new physiological data can be stored.

### Medical Devices

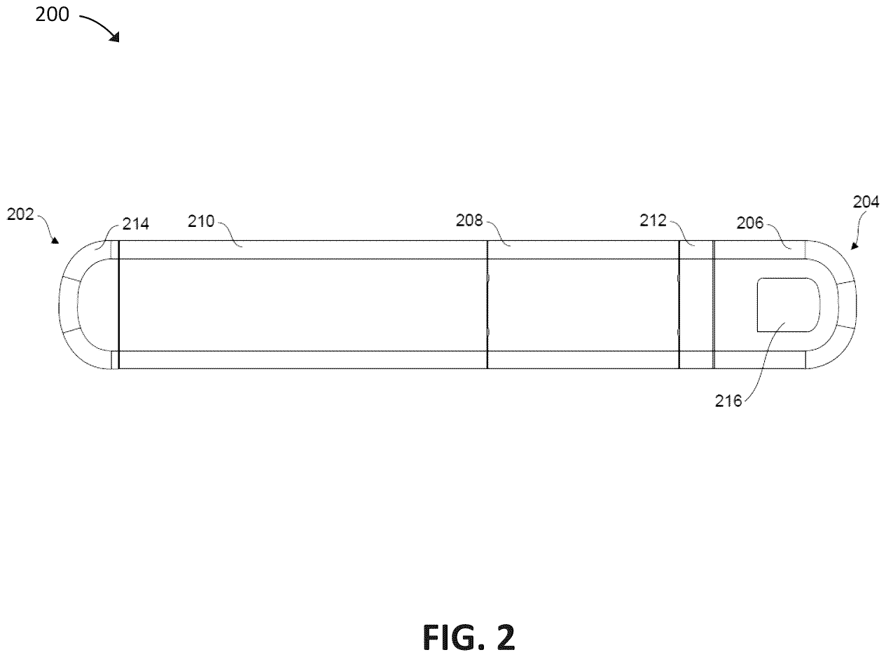

FIG. 2 is a side view of an implantable medical device 200 (hereinafter “IMD 200” for brevity). The IMD 200 may be, or may be similar to, the medical device 100 depicted in FIG. 1 and may be used in the system 100 of FIG. 1. Although the IMD 200 is implantable, other types of medical devices can include features described herein.

The IMD 200 includes an external housing that extends between a first end 202 and a second end 204. In the example of FIG. 2, the IMD 200 includes a first housing section 206, a second housing section 208, a third housing section 210, a fourth housing section 212, a first electrode 214, and a second electrode 216. Each of the housing sections can be separate components that are assembled together during manufacturing to create the external housing of the IMD 200. When assembled together, the housing sections can create a hermetically sealed enclosure. Although four separate housing sections are shown in FIG. 2, additional or fewer separate sections can be used to create the IMD 200. For example, instead of manufacturing separate housing sections and then assembling them together, the IMD 200 can be formed by molding (e.g., using a polymer material), casting, etc. And one or more of the housing sections can comprise a ceramic material.

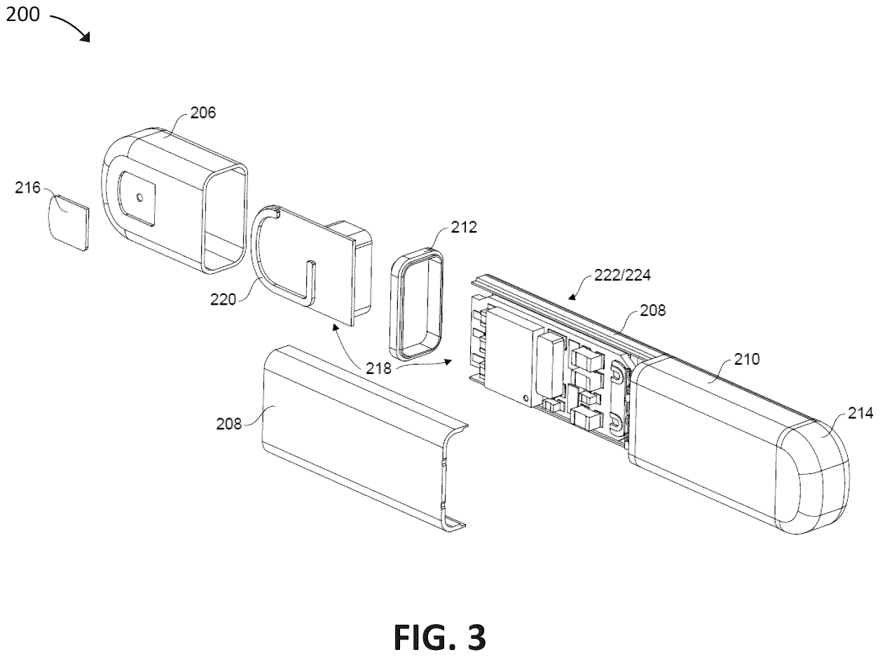

FIG. 3 shows a partially exploded view of the IMD 200. Once assembled, the second electrode 216 is coupled to the first housing section 206. In certain instances, the first housing section 206 comprises a ceramic material.

The third housing section 210 can comprise or can be a battery assembly (which may include one or more batteries). The exterior of the battery assembly can form the third housing section 210 in which one or more batteries (e.g., single-use battery cells, rechargeable battery cells) are positioned. The first electrode 214 is disposed at an end of the third housing section 210. In certain instances, the first electrode 214 is integrated with the battery assembly.

The fourth housing section 212 can function as an interface or coupler between the first housing section 206 and the second housing section 208. For example, the fourth housing section 212 can be used to couple the first housing section 206 to the second housing section 208. More specifically, in instances where the fourth housing section 212 comprises a metal such as titanium, the fourth housing section 212 can be assembled to the second housing section 208 via welding (e.g., laser welding). And, in instances where the first housing section 206 comprises a ceramic, the fourth housing section 212 can be brazed to the first housing section 206. In such instances, the fourth housing section 212 can be coupled between the first housing section 206 and the second housing section 208. As shown in FIG. 3, the fourth housing section 212 can be shaped as a continuous ring with an opening therethrough. Further, the fourth housing section 212 can include joint features such as one or more thinned sections or flange sections such that connecting the fourth housing section 212 to the other sections (e.g., via welding and/or brazing) can be accomplished. For example, portions of the other housing sections can overlap with the thinned or flanged sections of the fourth housing section 212 to provide overlapping surface area.

FIG. 3 shows a circuit board 218. For purposes of illustrating the various components of the IMD 200 in an exploded view, the circuit board 218 is shown as two separate components, but the two components can form a single circuit board. The circuit board 218 can comprise a rigid circuit board or a flexible circuit (e.g., a flexible circuit comprising polyimide, etc.).

An antenna 220 is coupled to the circuit board 218. Various other electrical components can be coupled to the circuit board 218. For example, the medical device 200 can include one or more memory components 222 coupled to the circuit board 218. The memory 222 can include a temporary memory (e.g., random access memory (RAM), cache memory) and longer-term memory (e.g., flash memory). In some instances, the temporary memory and the longer-term memory are separate sections or modules of a single chip package. In other instances, the temporary memory and the longer-term memory are separate components that are separately attached to the circuit board 218. In certain instances, the temporary memory is operated as a memory (e.g., a FIFO buffer memory) that automatically deletes (or overwrites) old data as new data is stored to the temporary memory—whereas the longer-term memory is not operated to automatically delete (or overwrite) old data but, instead, manages its storage capacity according to an operating mode of the IMD 200 (e.g., default operating mode versus non-default operating mode). In certain instances, the longer-term memory has a greater storage capacity (e.g., a greater amount of bytes, kilobytes, megabytes, gigabytes, and the like) than the storage capacity of the temporary memory.

Other electrical components can be coupled to the circuit board 218 too such as one or more integrated circuits (e.g., application specific integrated circuits, field-programmable gate arrays) programmed to perform functions such as processing and/or communication functions of the IMD 200. For example, the electrical components like the integrated circuits can include one or more processors 224 (e.g., microprocessors) coupled to the memory 222 or other memory components with instructions (e.g., in the form of firmware, and/or software) for performing functions of the IMD 200. Example functions of the circuitry include processing physiological data (e.g., converting sensed electrical signals from the electrodes into ECG data, detecting potential cardiac events based on the sensed electrical signals, saving physiological data to the memory, and the like). The electrical components can be electrically coupled to the one or more batteries such that the electrical components are powered by the one or more batteries.

### Methods for Programming Medical Devices

As noted above, recording and transmitting physiological data every time an event occurs can be an inefficient use of medical devices' power and memory capacity. Further, medical devices may have a limited overall memory capacity due to space and power constraints. To save power and memory capacity, medical devices may have a default operating mode that utilizes various power-saving and memory-saving approaches. In some instances, in a default operating mode, medical devices are programmed to transfer recorded physiological data only once per day or twice per day and limit how much physiological data is saved to memory for each detected event. For example, for a detected event, medical devices may default to storing only 1 minute of physiological data or only 30 seconds of physiological data before the onset of the detected event and 30 seconds of physiological data after the end of the detected event.

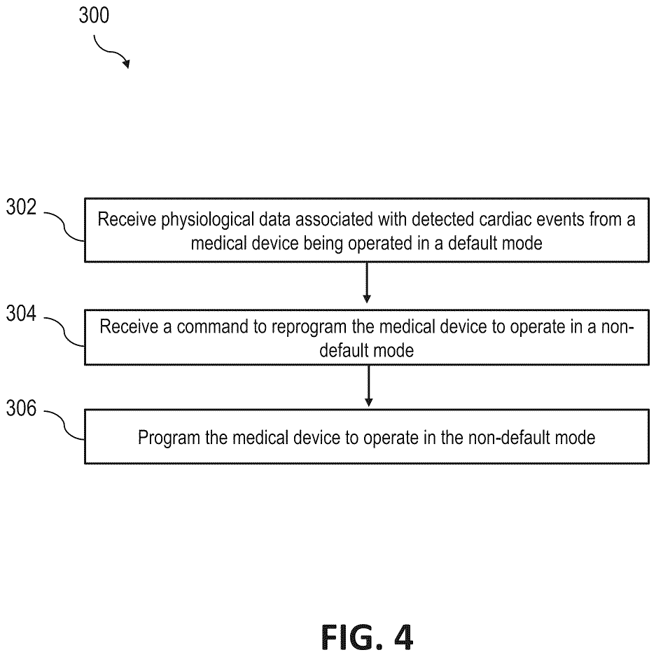

However, in certain situations, medical devices may be programmed to use a non-default operating mode. FIG. 4 shows an outline of steps of a method 300 for modifying a medical device's operating mode. The method 300 is described from the perspective of a device that causes the medical device (e.g., such as the medical devices described herein) to be reprogrammed. The device can be a device such as the receiver 106 of FIG. 1, including but not limited to a mobile computing device such as a programmer, smartphone, tablet computer, laptop computer, etc. The receiver 106 can operate an application (e.g., a software-based application operated by the receiver 106) that enables the receiver 106 to communicate with medical devices. And the receiver 106 can receive commands from a remote system such as the computing systems 108/110 of FIG. 1.

The method 300 includes receiving physiological data associated with detected cardiac events from a medical device (e.g., an implantable medical device) being operated in a default mode (block 302 of FIG. 4). A device such as the receiver 106 of FIG. 1 can receive the physiological data (e.g., ECG data) from the medical device.

The detected cardiac events can be based on physiological data such as ECG data. The circuitry of the medical device can be programmed to compare the sensed ECG data to various thresholds set to identify cardiac events like bradycardia, tachycardia, pause, atrial tachycardia, atrial fibrillation, PVCs, among others. For example, potential bradycardia events can be based on a person's heart rate dropping below a threshold (e.g., 60 bpm, 50 bpm, 40 bpm, 30 bpm) for a certain period of time (e.g., 1 second, 2 seconds, 3 seconds, 4 seconds). As another example, potential tachycardia events can be based on a person's heart rate rising above a threshold (e.g., 140 bpm, 150 bpm, 160 bpm, 170 bpm) for a certain period of time (e.g., 3 seconds, 4 seconds, 5 seconds, 6 seconds, 7 seconds). As another example, potential pause events can be based on a beat-to-beat time rising above a threshold (e.g., 2 seconds, 3, seconds, 4 seconds, 5 seconds). As another example, potential atrial tachycardia events can be based on a person's average heart rate rising above a threshold (e.g., 140 bpm, 150 bpm, 160 bpm, 170 bpm) over a certain period of time (e.g., 1 hour, 2 hours, 3 hours, 4 hours, 5 hours). As another example, potential atrial fibrillation events and PVC events can be based on detecting that such events have occurred more than a threshold number of times over a period of time.

The method 300 further includes receiving a command to reprogram the medical device to operate in a non-default mode (block 304 of FIG. 4). A device such as the receiver 106 of FIG. 1 can receive the command. In some instances, commands can be inputted (e.g., manually inputted) to a user interface of the receiver 106. In other instances, commands can be generated by the computing system 108/110 and transmitted to the receiver 106. For example, a patient's physician may initiate the command from one of the computing systems 108/110, and the command is transmitted to a device able to reprogram the medical device.

The method 300 further includes programming the medical device to operate in the non-default mode (block 306 in FIG. 4), in response to the command. Programming the medical device to operate in the non-default mode can include transmitting a command from the receiver 106 to the medical device that causes the medical device to change operating modes.

In the non-default operating mode, the amount of physiological data wirelessly transmitted by the medical device to the receiver 106 (or another device) can increase. This can be accomplished in multiple ways.

As one example, in the non-default operating mode, a length (e.g., an amount of time) of physiological data stored (e.g., to longer-term memory) for each detected cardiac event can be increased compared to the length stored for detected cardiac events in the default operating mode. In some instances, the default operating mode may automatically store ˜1 minute of physiological data for each detected cardiac event, whereas in the non-default operating mode, the length can be increased (e.g., to 1.5-4 minutes such as 2 minutes, 3 minutes, 4 minutes). In other instances, the length is decreased such that more events (and associated physiological data) are stored to local memory given the fixed capacity of the memory. In some instances the default operating mode may automatically store physiological data from between the onset and end of the detected cardiac event as well as 30 seconds before and after the onset and end. In the non-default operating mode, the amount of physiological data before and after the onset and end can be increased (e.g., to 45 second-2 minutes such as 1 minute, 2 minutes).

As another example, in the non-default operating mode, physiological data can be continuously stored to memory (e.g., to longer-term memory) regardless of whether a detected cardiac event has occurred, and all stored physiological data can be transmitted to the receiver 106 (or another device). The frequency at which the physiological data is transferred can depend on the capacity of the memory. If the memory can only store ˜30 minutes of physiological data, then the medical device can be programmed (in the non-default operating mode) to transmit the physiological data every 20-30 minutes (e.g., 20 minutes, 25 minutes, 30 minutes). Once the physiological data is transferred, the physiological data can be deleted from the memory of the medical device to allow for new physiological data to be stored to the memory. Alternatively, the medical device could be programmed (in the non-default operating mode) to continuously transfer physiological data to the receiver 106 (or another device).

As another example, in the default operating mode, any physiological data stored for detected cardiac events may be transmitted to the receiver 106 (or other device) only once per day. In the non-default operating mode, the frequency at which the stored physiological data is transmitted can increase. Instead of once-per-day, any physiological data (that has not already been transferred) can be transferred from the medical device to the receiver 106 (or other device) continuously or every 10 minutes, 20 minutes, 30 minutes, hour, 2 hours, 6 hours, 12 hours. The frequency at which physiological data is transferred can depend on the time of day and activity levels (e.g., higher frequency during the day or when the patient is active compared to night or when the patient is resting or otherwise inactive). As such, the medical device may include a sensor (e.g., an activity sensor such as an acceleration sensor, a posture sensor) that can sense a patient's movement and/or posture. Once the physiological data is transferred, the physiological data can be deleted from the memory of the medical device to allow for new physiological data to be stored to the memory.

As another example, in the default operating mode, storing physiological data associated with more-critical cardiac events may be prioritized for storage and transmission over less-critical cardiac events. In the non-default operating mode, all detected cardiac events can be stored to memory instead of storing only higher-priority cardiac events.

As another example, in the non-default operating mode, the medical device may use thresholds that are more sensitive for detecting potential cardiac events. For example, the minimum or maximum heart rate, duration, etc., thresholds for detecting a potential cardiac event can be modified such that more potential cardiac events are detected compared to the thresholds used in the default operating mode.

As another example, in the default operating mode, the medical device may have a maximum number of detected cardiac events that are stored to memory and transmitted. In the non-default operating mode, the maximum number can be increased, or the maximum number requirement can be removed altogether.

As another example, in the non-default operating mode, the medical device may modify the trigger for when transmission of the physiological data associated with a detected cardiac event is initiated. For example, instead of transmitting physiological data once-per-day or at only specific times, the medical device can be programmed to transmit physiological data whenever a potential cardiac event has been detected, when a certain type of potential cardiac event has been detected, and/or when a heart rate associate with a detected cardiac event is above or below a threshold.

As another example, in the non-default operating mode, the medical device may increase an amount of non-ECG data (e.g., non-ECG diagnostic data) that is stored and transmitted. For example, the medical device may have sensors such as acceleration sensors, chemical sensors, optical sensors, posture sensors, etc. Output data from one or more of these non-ECG sensors can be stored and transmitted with the physiological data—regardless of whether the output data is associated with a detected cardiac event.

Initiating reprogramming of the medical device can be based on various factors or scenarios. As one example, the process of programming the medical device to operate in the non-default operating mode can be based on a physician initiating the change in programming. This may be based on a change in drug regimen, following an ablation procedure, following implantation of a stent, etc. As another example, the process of programming the medical device to operate in the non-default operating mode can be based on a patient having reported a certain number of symptoms (e.g., via a software application). In this scenario, the patient may use an application on their smartphone (or another device) to input when they feel symptoms, and the number of symptoms (e.g., over a certain period of time) can trigger the process of programming the medical device to operate in the non-default operating mode.

In certain instances, the medical device can automatically revert back to the default operating mode after a predetermined period of time (e.g., 2 days, 3, days, 4 days, 5 days, 6 days, 7 days, or more). Reverting back to the default operating mode can occur without additional interaction or commands from the receiver 106 (or another device). In other instances, the medical device will continue to operate in the non-default operating mode until it receives a command to revert back to the default operating mode.

In certain instances, the non-default operating mode includes preprogrammed modifications to the items described herein (e.g., increased length of physiological data for each detected event, maximum number of stored detected events, frequency of transmission of physiological data). In other instances, the changes associated with the non-default operating mode can be customized by a physician. In this instances, the command may include instructions to modify specific parameters of the default operating mode.

As noted above, the medical device may be programmed to transmit (e.g., wirelessly transmit) any physiological data recorded to longer-term memory periodically to another device such as the receiver 106. The physiological data may ultimately be transmitted to a clinic for review by a physician.

In certain instances, before the physiological data is transmitted to a clinic, data such as ECG data is transmitted and processed by a computing system such as a server. Because the server has more computational and memory capacity than a medical device, the server can provide a second review of the ECG data before clinical review. The server can be programmed to filter out non-episodes and noise, to confirm episodes, and/or to determine confidence values for episodes, among other functions.

To assist with the functions described immediately above, the server can include one or more machine learning models (e.g., types of deep neural networks) to process the ECG data to classify cardiac activity. For example, the machine learning model may compare the ECG data to labeled ECG data to determine which labeled ECG data the ECG data most closely resembles. The labeled ECG data may identify a particular cardiac event such as a PVC episode. In certain instances, the machine learning model includes two paths, where the first path is a deep convolutional neural network and the second path is a deep fully-connected neural network. The deep convolutional neural network receives one or more sets of beats (e.g., beat trains with 3-10 beats) which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, and a non-linear activation function layer. This set of layers can be repeated multiple times.

The deep fully connected neural network can receive RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

If the machine learning model determines that the ECG data most closely resembles a labeled ECG data associated with a cardiac event, then the machine learning model may determine that the patient has experienced that cardiac event. Additionally, the machine learning model may measure or determine certain characteristics of the cardiac activity of the patient based on the ECG data. For example, the machine learning model may determine a heart rate, a duration, or a beat count of the patient during the cardiac event based on the ECG data. The server stores the cardiac event and associated metadata such as information like beat classification, heart rate, duration, beat count, etc., in a database for storage.

Outputs of the machine learning model and the underlying ECG data can be transmitted to a clinic for physician review.

### Computing Devices and Systems

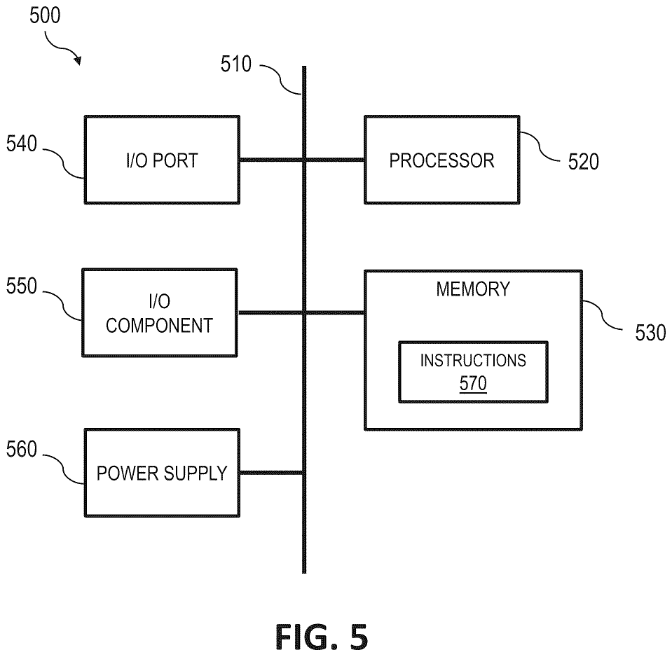

FIG. 5 is a block diagram depicting an illustrative computing device 500, in accordance with instances of the disclosure. The computing device 500 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 5 and described below. For example, the medical devices, receivers, and computing systems can each include their own set (or partial set) of components shown in FIG. 5 and described below.

In instances, the computing device 500 includes a bus 510 that, directly and/or indirectly, couples one or more of the following devices: a processor 520, a memory 530, an input/output (I/O) port 540, an I/O component 550, and a power supply 560. Any number of additional components, different components, and/or combinations of components may also be included in the computing device 500.

The bus 510 represents what may be one or more busses (such as, for example, an address bus, data bus, or combination thereof). Similarly, in instances, the computing device 500 may include a number of processors 520, a number of memory components 530, a number of I/O ports 540, a number of I/O components 550, and/or a number of power supplies 560. Additionally, any number of these components, or combinations thereof, may be distributed and/or duplicated across a number of computing devices.

In instances, the memory 530 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 530 stores computer-executable instructions 570 for causing the processor 520 to implement aspects of instances of components discussed herein and/or to perform aspects of instances of methods and procedures discussed herein. The memory 530 can comprise a non-transitory computer readable medium storing the computer-executable instructions 570.

The computer-executable instructions 570 may include, for example, computer code, machine-useable instructions, and the like such as, for example, program components capable of being executed by one or more processors 520 (e.g., microprocessors) associated with the computing device 500. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 570 may be configured to be executed by the processor 520 and, upon execution, to cause the processor 520 to perform certain processes. In certain instances, the processor 520, memory 530, and instructions 570 are part of a controller such as an application specific integrated circuit (ASIC), field-programmable gate array (FPGA), and/or the like. Such devices can be used to carry out the functions and steps described herein.

The I/O component 550 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), via Bluetooth, a cellular data network, via the internet using an internet service provider, and the like.

Aspects of the present disclosure are described with reference to block diagrams of methods, devices, systems and computer program products. It will be understood that each block of the block diagrams, and combinations of blocks can be implemented by computer program instructions.

Various modifications and additions can be made to the exemplary instances discussed without departing from the scope of the present invention. For example, while the instances described above refer to particular features, the scope of this invention also includes embodiments having different combinations of features and instances that do not include all of the described features. Accordingly, the scope of the present invention is intended to embrace all such alternatives, modifications, and variations as fall within the scope of the claims, together with all equivalents thereof.

We claim:

1. A method comprising:
receiving physiological data associated with detected cardiac events from an implantable medical device being operated in a default mode;
receiving a command to reprogram the implantable medical device to operate in a non-default mode; and
programming the implantable medical device, using a remote device, to operate in the non-default mode,
wherein the programming increases an amount of physiological data wirelessly transmitted by the implantable medical device compared to the default mode.

2. The method of claim 1, wherein the programming increases a length of physiological data stored for each detected cardiac event.

3. The method of claim 1, wherein the programming increases a frequency at which the physiological data is wirelessly transmitted.

4. The method of claim 1, wherein the physiological data is deleted from memory after being wirelessly transmitted by the implantable medical device.

5. The method of claim 1, wherein the programming adjusts prioritization of storage and deletion of the physiological data.

6. The method of claim 1, wherein the programming increases a maximum number of cardiac events stored to memory.

7. The method of claim 1, wherein the physiological data is wirelessly transmitted to the remote device.

8. The method of claim 1, wherein the programming changes a trigger for initiating transmission of the physiological data.

9. The method of claim 1, wherein the programming changes an amount of time stored to memory before an onset of a cardiac event and an end of the cardiac event.

10. The method of claim 1, wherein the programming increases diagnostic data associated with each cardiac event.

11. The method of claim 1, further comprising:
programming the implantable medical device to revert back to the default operating mode after a predetermined period of time.

12. The method of claim 1, wherein, in the default mode, the medical device is programmed to transfer physiological data only once or twice per day.

13. A system comprising:
a programmer comprising a user interface, memory, and one or more processors, wherein the memory comprises instructions that, when executed by the one or more processors, cause the programmer to:
receive a command to reprogram an implantable medical device to change operating from a default mode to a non-default mode, and
in response to the command, cause the implantable medical device to be programmed to operate in the non-default mode,
wherein the non-default mode increases an amount of physiological data wirelessly transmitted by the implantable medical device compared to the default mode.

14. The system of claim 13, wherein, in the non-default mode, a length of physiological data stored for each detected cardiac event is increased.

15. The system of claim 13, wherein, in the non-default mode, a frequency at which the physiological data is wirelessly transmitted is increased.

16. The system of claim 15, wherein the frequency is greater than twice per 24-hour period.

17. The system of claim 13, wherein the non-default mode changes an amount of time stored to memory before an onset of a cardiac event and an end of the cardiac event.

18. The system of claim 17, wherein the amount of time is 45 seconds to 2 minutes.

19. The system of claim 13, further comprising:
the implantable medical device, wherein the implantable medical device includes electrodes.

20. The system of claim 19, further comprising:
a remote computing system, wherein the command is initiated at the remote computing system.
